(19) United States
(12) Patent Application Publication
(10) Pub. No.: US 2002/0132279 A1
(43) Pub. Date: Sep. 19, 2002

(54) Formula to manipulate blood glucose via the calculated ingestion of carbohydrate

(76) Inventor: Linda Hockersmith, Scottsdale, AZ (US)

Correspondence Address: GLENN PATENT GROUP, 3475 EDISON WAY, SUITE L, MENLO PARK, CA, 94025, US

(21) Appl. No.: 09/766,427

(22) Filed: Jan. 18, 2001

Related U.S. Application Data
(60) Provisional application No. 60/208,027, filed on May 30, 2000.

(51) Int. Cl. C12Q 1/66; C12Q 1/54; G06F 19/00

(52) U.S. Cl. 435/14; 702/19

(57) ABSTRACT

A formula for calculating the amount of carbohydrate necessary to achieve a target blood glucose excursion in a diabetic test subject is based on a baseline blood glucose level, a target level to be achieved and an index of the subject's sensitivity to carbohydrate. Initially, the index value is an exemplary value based on typical carbohydrate sensitivities displayed by various types of diabetics. However, the index may be individualized to a test subject based on an actual glucose excursion.

A method of effecting a shift in blood glucose level in a diabetic subject incorporates the formula presented above. Furthermore, a method for dietary management of a diabetic individual's glycemic profile, wherein an optimal glycemic profile is achieved and maintained, also incorporates the formula.

### CROSS-REFERENCE TO RELATED APPLICATION

This application claims benefit of U.S. Provisional Patent Application Serial No. 60/208,027, filed on May 30, 2000.

### BACKGROUND OF THE INVENTION

1. Field of the Invention

The invention relates to measurement of blood glucose levels. More particularly, the invention relates to a formula and method for achieving a targeted response in test subjects' blood glucose levels from the ingestion of a quantified amount of carbohydrate, thereby enabling a non-significant risk approach to obtaining a variety of glycemic profiles.

2. Description of Related Art

The increase in blood sugar levels resulting from the ingestion of carbohydrate foods has long been known; in fact it is of ongoing concern in those afflicted with diabetes mellitus. Furthermore, carbohydrate intolerance is one of the major criteria for a diagnosis of diabetes mellitus. The Oral Glucose Tolerance test employs ingested carbohydrate in a predetermined form and amount to quantify a test subject's response to a resulting glucose challenge. See Oral glucose tolerance test, Complete Guide to Medical Tests,

http://www.healthgate.com/tests/tests/test240.shtml. Criteria have been established to evaluate this response according to the type of diabetes to be diagnosed. In the case of gestational diabetes, a blood glucose level exceeding 180 mg/dl is indicative of an impaired insulin response and therefore suggestive of diabetes. See Oral glucose tolerance test for gestational diabetes,

http://www.medstudents.com/ginob/ginob4t1.htm. In the case of Type 1 or Type 2 diabetes, a blood glucose level exceeding 200 mg/dl is indicative of an impaired insulin response. While the blood glucose excursion may fall back to normal over a period of time, the Oral glucose tolerance test is concerned only with the peak blood level of glucose. It does not concern itself with the rate of change in glucose levels or the amount of time it takes for glucose levels to fluctuate from a high point to a low point.

A liquid carbohydrate beverage such as GLUCOLA is employed in a conventional Glucose Tolerance test. Unfortunately, such glucose beverages have met with poor patient acceptance, often causing nausea, or even vomiting.

In addition to the above-mentioned carbohydrate beverage, alternative carbohydrate sources have been proposed, for example, a predetermined number of jellybeans, or SUSTACAL, a liquid food supplement. See A jelly bean glucose test, http://www.childbirth.org/articles/jellybean.html. However, the medical community has been slow to adopt the use of alternate carbohydrate sources in diagnostic procedures.

Glucose excursions are often induced through the intravenous administration of dextrose, a disaccharide composed of two glucose subunits, during procedures commonly known as euglycemic insulin clamp techniques. Over the course of a procedure of this type, exogenous insulin may be infused at a rate that maintains a constant plasma insulin level above a fasting level. The glucose infusion is delivered via an indwelling catheter at a rate based on plasma glucose measurements done at five-minute intervals. When the plasma glucose level falls below basal level, the glucose infusion rate is increased to return plasma to basal levels. Conversely, glucose infusion is decreased, or the insulin infusion is increased when plasma glucose exceeds basal levels. The total amount of glucose infused over time, or the M value, comprises an index of insulin action on glucose metabolism. See Consensus development conference on insulin resistance, Diabetes Care, vol. 21 (2) p. 310 (1998). A typical profile resulting from this procedure would resemble a straight line, but a stepped increase or decrease in blood glucose may also be obtained. See Preservation of physiological responses to hypoglycemia two days after antecedent hypoglycemia in patients with IDDM, Diabetes Care, vol. 20 (8) p. 1293 (1997). Although euglycemic clamp studies are effective for quantifying the amount of insulin required to achieve a particular glycemic pattern, they suffer the disadvantage of being highly impractical in clinical settings. Additionally, they entail a significant amount of risk to the patient, and they generally meet with poor patient acceptance.

Controlling a patient's intake of carbohydrate has long played an important role in the dietary management of a variety of health conditions. One such approach, carbohydrate counting, has become popular in diabetes control. See Carbohydrate counting: a new way to plan meals,

http://www.diabetes.com/health library/articles/13t103235.html. Using such methods, the total dietary requirement for carbohydrate may be calculated and distributed throughout the day's meals and snacks, thus allowing many to achieve better control over their diabetes.

The glycemic index provides a way to quantify the effect of a type of carbohydrate on glucose excursion, resulting in better diabetes control. See The glycemic index: another option for managing diet,

http://www.diabetes.com/health library/articles/13t103210.html.

Carbohydrate sources with a high glycemic index produce a correspondingly greater increase in blood glucose level than those carbohydrates having a lower glycemic index. For example, a baked potato has a high index, while low-fat yogurt or rice bran have relatively low indexes. Thus, a baked potato produces a greater increase in blood glucose level than the yogurt or rice bran. While the glycemic index is a useful tool for predicting a glucose excursion, it is not concerned with inducing predetermined glycemic profiles, particularly not profiles having more than one glucose excursion.

Counting the total amount of carbohydrate in a meal allows the diabetic to calculate a compensatory insulin bolus more accurately. See Carbohydrate counting, http://www.minimed.com/files/mmn029.htm. However, such dietary controls and formulas serve to diminish glycemic response rather than to target a predetermined glycemic profile.

Management of carbohydrate intake is a common feature in weight management programs. The positive impact of both low and high-carbohydrate diets in weight reduction programs is well known. Controlling carbohydrate intake affects total calorie intake, appetite, water loss and many other factors in this multivariate problem. In fact, engineered food sources that affect the rate at which carbohydrate is digested or eliminated are available. While these carbohydrate control rationales do achieve a reduction in impact on blood glucose level and calorie metabolism, they do not serve as purposeful predictors of glycemic profiles.

### SUMMARY OF THE INVENTION

The invention provides a method for calculating the required amount of carbohydrate to ingest orally to achieve a target blood glucose excursion in a diabetic test subject. The invented method is based on a baseline blood glucose level, a target level to be achieved and a novel numerical index that quantifies the subject's sensitivity to carbohydrate. Initially, the index value is a generalized value based on typical carbohydrate sensitivities displayed by various types of diabetics. However, the index may be individualized to a test subject based on an actual glucose excursion. A method of effecting a shift in blood glucose level in a diabetic subject incorporates the formula presented above. Furthermore, a method for dietary management of a diabetic individual's glycemic profile, wherein an optimal glycemic profile is achieved and maintained, also incorporates the formula.

### BRIEF DESCRIPTION OF THE DRAWINGS

FIG. 1 shows a first pair of anti-correlated glycemic profiles, according to the invention;

FIG. 2 shows a second pair of anti-correlated glycemic profiles, according to the invention;

FIG. 3 shows a targeted glycemic profile for a first calibration visit superimposed on actual measured glycemic profiles from a subject pool, according to the invention;

FIG. 4 shows a targeted glycemic profile for a second calibration visit superimposed on actual measured glycemic profiles from a subject pool, according to the invention;

FIGS. 5-8 each show measured glycemic profiles for first and second calibration visits imposed on one another for first, second, third and forth subjects, respectively, according to the invention; and

FIGS. 9-12 each show a measured glycemic profile for a third calibration visit for first, second, third and fourth subjects respectively, according to the invention.

### DETAILED DESCRIPTION

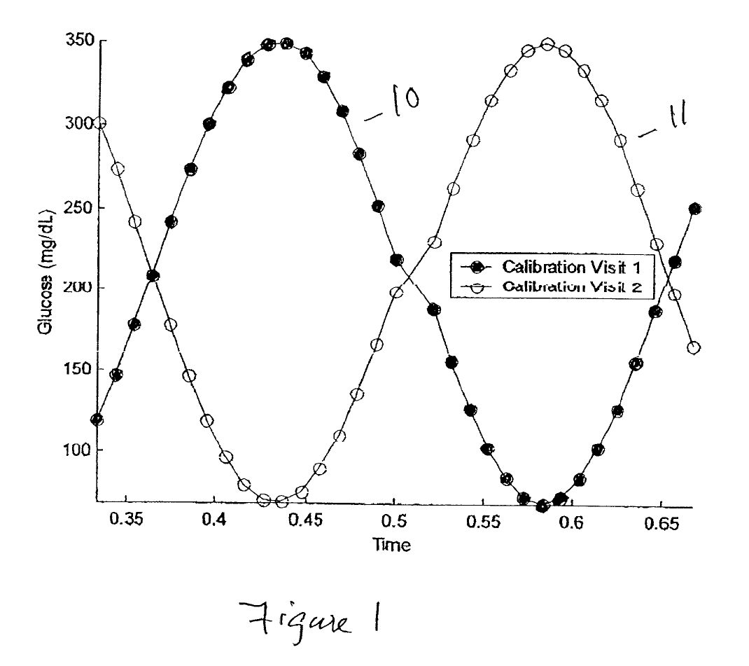

Calibrating a noninvasive blood glucose monitor to an individual necessitates a calibration that is correlated only to blood glucose. Generating such a calibration requires reference blood glucose values that are uncorrelated to sampling factors such as skin temperature, environmental temperatures, time of day, and other blood analytes. FIG. 1 shows a pair of targeted, anti-correlated glycemic profiles 10, 11 in which one profile is the inverse of the other. The invention provides a method of calibrating a noninvasive blood glucose monitor using blood glucose reference values, in which correlation to the sampling factors previously mentioned is greatly reduced or eliminated. A test subject's blood glucose levels are actively controlled or manipulated through the oral ingestion of carbohydrate foods and the administration of rapid-acting insulin in such a way that the patterns of the targeted glycemic profiles of FIG. 1 are reproduced by the subject's own glycemic profile during successive calibration visits. Thus, since the subject's blood glucose level is under active control, the influence of other sampling factors on the reference values is greatly reduced or eliminated. By using anti-correlated profiles in separate calibration visits, the influence of factors that correlate across visits is reduced.

In general the various steps of the invented method are:

manipulating a subject's blood glucose level such that patterns of the profiles are reproduced by subject's own glycemic profile;

performing reference blood glucose measurements at predetermined intervals;

gathering non-invasive spectral measurements with a non-invasive glucose measurement instrument at said predetermined intervals; and

generating a calibration that correlates reference measurements and spectral measurements, such that an algorithm predicts a blood glucose level from a new spectral sample.

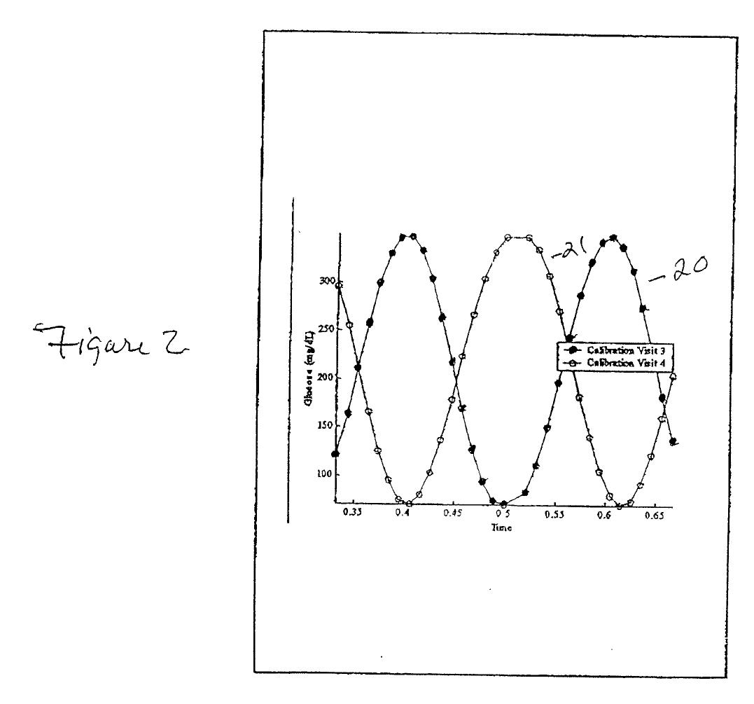

In a preferred embodiment, the invention utilizes the targeted profiles of FIG. 1, involving a single glucose excursion. A subject makes two calibration visits, lasting approximately eight hours each. The first profile is produced on the first visit and the second profile is produced on a second visit. In an alternate, equally preferred embodiment, the invention utilizes the profiles shown in FIG. 2. The profiles 20, 21 involve multiple glucose excursions. As with the previous embodiment of the invention, two calibration visits are required. In a third, equally preferred embodiment of the invention, the profiles of both FIG. 1 and FIG. 2 are employed in the calibration method. In this case, four calibration visits are required.

Throughout the duration of each calibration visit, the subject's blood glucose level is measured at regular intervals using conventional invasive methods. Concurrently, noninvasive spectral measurements are taken.

The subject is fed either carbohydrate-rich meals to produce a glucose excursion, or low-carbohydrate meals to promote a drop on blood sugar level. The amount of carbohydrate to be ingested is calculated according to an inventive formula, described in greater detail below. The formula, based on a current glucose level, a target glucose level and the subject's sensitivity to carbohydrate, utilizes a novel numerical index to quantify carbohydrate sensitivity. Meals are composed of carefully selected, conventional foods and beverages. Orally ingesting carbohydrate in the form of conventional foods and beverages provides several important advantages. It provides a closer approximation of the subject's daily routine than conventional methods of inducing a glucose excursion do. In addition, ingesting the carbohydrate orally, rather than having it administered through intravenous infusion, as is often done, greatly diminishes any risk to the subject from the IV, and the glucose excursion resulting. Test subjects find the conventional foods and beverages to be much more palatable than the liquid glucose beverages often used to induce glucose excursions. The beverages, unpleasantly sweet, often induce nausea and even vomiting. While ingestion of the required amount of carbohydrate easily produces the required glucose excursion, a corresponding drop in blood sugar within the required time period requires the administration of insulin. Rapid-acting insulin, such as HUMALOG, produced by Eli Lilly & Co. of Indianapolis, Ind. is employed to produce the necessary drop in blood sugar level.

The blood glucose reference values and the spectral measurements furnish a data set upon which the calibration is based. The data are first divided into a calibration data set and a test set. The reference values and the spectral measurements are correlated using commonly known multivariate techniques. An algorithm is generated, also using conventional analytical methods, based on the calibration data set, that predicts a blood glucose level from a new spectral measurement. The various aspects of the invention, particularly the method of producing targeted fluctuations in the subject's blood glucose level are described in greater detail below.

Experiment: A study was performed to determine if a targeted response in blood glucose level could be achieved from the oral ingestion of a calculated amount of carbohydrate in both Type 1 and Type 2 diabetic subjects. Use of a carbohydrate formula to calculate the required amount of carbohydrate would allow a low risk approach to obtaining a variety of predetermined glycemic profiles, which could subsequently be used to develop single subject glucose calibrations for noninvasive instrumentation.

In order to provide a broad range of reference glucose values, a target glucose profile for each calibration visit was specified as a glucose level range of from less than 90 mg/dL through a targeted high of greater than 300 mg/dL for each calibration visit, with a rate of change <5 mg/dl/minute. As previously explained, it was necessary to obtain data sets in which the patterns resulting from the blood glucose reference values did not correlate across calibration visits; in other words, they were to be very dissimilar to each other. Therefore, the glycemic profiles were to be anti-correlated pairs; that is, one profile of a pair was to be the inverse of the other profile of the pair. During a first calibration visit, a glucose excursion that mimicked the first profile of a pair was to be achieved. The goal for a second visit was to achieve a glucose excursion that mimicked the second profile of the pair. Both calibration visits were eight hours in duration.

During the all-day calibration visits, the subjects were fed meals alternately composed of all carbohydrate or protein with non-digestible carbohydrate in order to achieve the recommended glucose profiles. The form of the carbohydrate was not limited, but was supplied both in the form of liquids and solid foods having a relatively low fat content. In addition, a rapid-acting insulin such as HUMALOG, manufactured by Eli Lilly and Co. of Indianapolis Ind., was employed to lower blood glucose levels, thus allowing the target profiles to be achieved in the allotted calibration time period.

Throughout each visit, non-invasive forearm scans were collected at fifteen-minute intervals using a near-infrared spectrometer instrument. Reference blood glucose measurements were done at the same time. For the invasive glucose determinations, capillary blood was collected from fingersticks and analyzed with a Hemocue Blood Glucose Analysis Instrument, manufactured by Hemocue AB of Ängleholm, Sweden.

The study participants were individuals diagnosed as having diabetes (Type I or II) who were well controlled, having HbA₁C (total glycosylated Hemoglobin) levels of less than 7.5%. Table 1, below, provides demographic information on the subject pool.

TABLE 1
Subject demographics

| | | | | | Year | | | |
|---|---|---|---|---|---|---|---|---|
| | | | | | of | | | |
| | | | | Dia- | Di- | | | |
| | | | Ethnic- | betes | ag- | Health | Pro- | |
| | Sex | DOB | ity | Status | nosis | Status | teinuria | A1C |
| 1 | F | 6/10/58 | HIS | 2 | 1991 | Good | 1+ | 7.4 |
| 2 | M | 11/08/6 | CAU | 2 | 1994 | Good | Neg | 6.9 |
| 3 | M | 01/23/4 | CAU | 2 | 1993 | Good | Neg | 6.0 |
| 4 | F | 06/26/0 | CAU | 1 | 1982 | Good | Neg | 6.0 |
| 5 | M | 08/23/3 | CAU | 2 | 1998 | Fair | Neg | 6.1 |
| 6 | M | 05/07/6 | CAU | 2 | 1999 | Good | 1+ | 6.5 |
| 7 | M | 01/18/7 | CAU | 2 | 1996 | Good | 2+ | 5.5 |
| 8 | F | 02/24/4 | CAU | 1 | 1964 | Good | Trace | 7.5 |
| 9 | F | 04/02/5 | HIS | 2 | 1994 | Good | Trace | 7.5 |
| 10 | F | 05/22/3 | CAU | 2 | 1998 | Good | Neg | 5.3 |

The formula used to calculate the amount of carbohydrate required to produce the desired glucose excursion is:

$$CHO = \frac{TARGET - STARTING}{X} , \qquad ( 1 )$$

where CHO is the amount of carbohydrate in grams, Target is the glucose level to be achieved, Starting is the current glucose level and X is a numerical index of the subject's sensitivity to carbohydrate challenge, described in greater detail below.

Table 2, below, shows a maximum and minimum, range and standard deviation of the glucose values for calibration visits of all clients. Maximum is the highest value achieved during a glucose excursion; minimum is a low value that may precede or follow a maximum value and the range is the span between maximum and minimum. As the results show, the target maximum and minimum values were achieved in ten out of twenty-three visits. Three subjects out of ten achieved the target range for both visits one and two.

TABLE 2
Glucose statistics for visits 1, 2, and 3
Entire Day

| | | | | | |
|---|---|---|---|---|---|
| Subject | Visit | Max | Min | Range | STD |

| | | | | | |
|---|---|---|---|---|---|
| 1 | 1 | 287 | 103 | 184 | 68.0 |
| 1 | 2 | 228 | 57 | 11 | 48.0 |
| 2 | 1 | 313 | 66 | 247 | 87.0 |
| 2 | 2 | 379 | 76 | 303 | 97.1 |
| 3 | 1 | 326 | 62 | 264 | 90.9 |
| 3 | 2 | 297 | 71 | 226 | 68.2 |
| 4 | 1 | 399 | 40 | 359 | 103.7 |
| 4 | 2 | 372 | 64 | 308 | 95.1 |
| 5 | 1 | 283 | 70 | 213 | 49.1 |
| 5 | 2 | 326 | 75 | 251 | 88.1 |
| 6 | 1 | 234 | 97 | 137 | 42.7 |
| 6 | 2 | 345 | 102 | 243 | 82.9 |
| 7 | 1 | 331 | 44 | 287 | 99.3 |
| 7 | 2 | 230 | 58 | 172 | 49.8 |
| 7 | 3 | 287 | 97 | 190 | 60.2 |
| 8 | 1 | 395 | 74 | 321 | 98.3 |
| 8 | 2 | 357 | 74 | 283 | 88.2 |
| 8 | 3 | 390 | 54 | 336 | 99.0 |
| 9 | 1 | 255 | 103 | 152 | 36.3 |
| 9 | 2 | 217 | 75 | 142 | 56.7 |
| 9 | 3 | 196 | 70 | 126 | 40.0 |
| 10 | 1 | 173 | 67 | 106 | 36.8 |
| 10 | 2 | 207 | 85 | 122 | 36.7 |

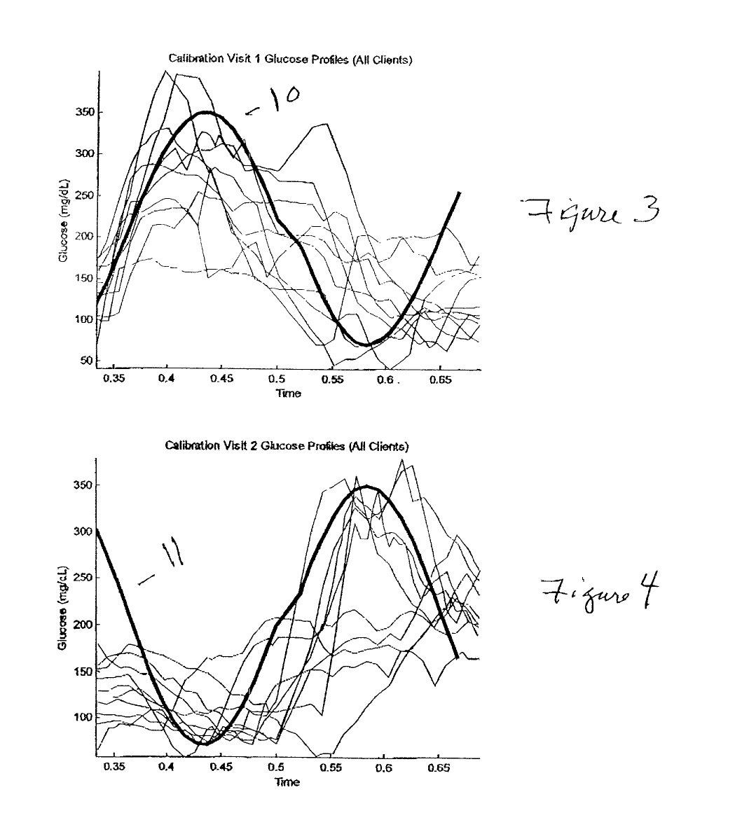

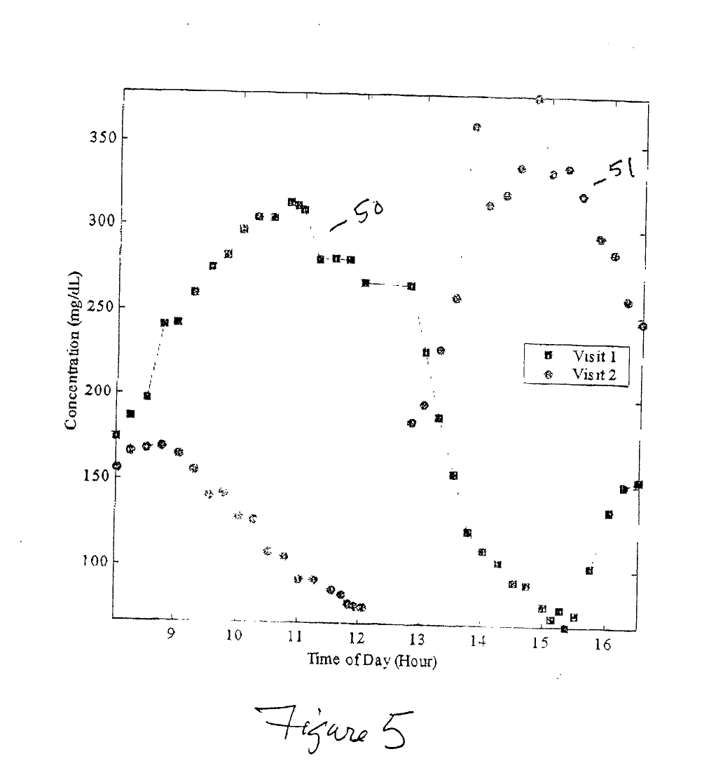

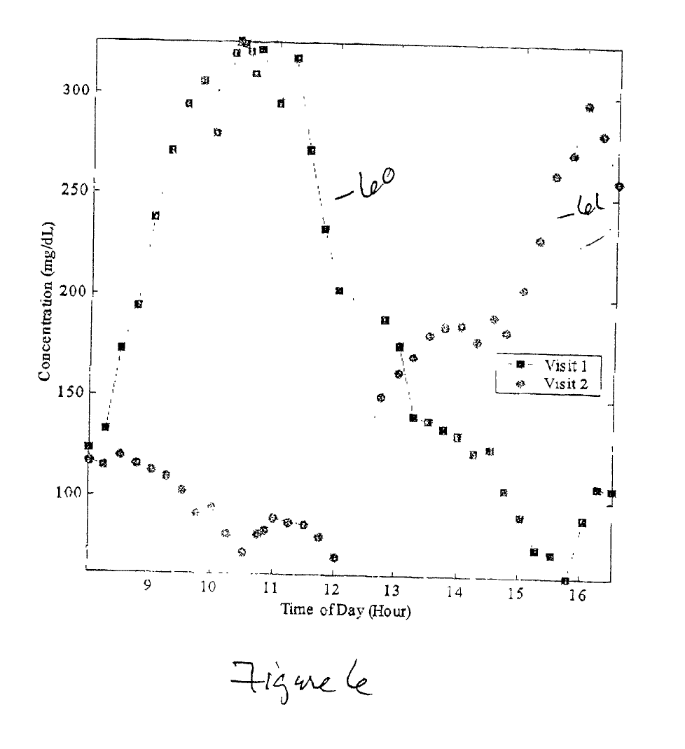

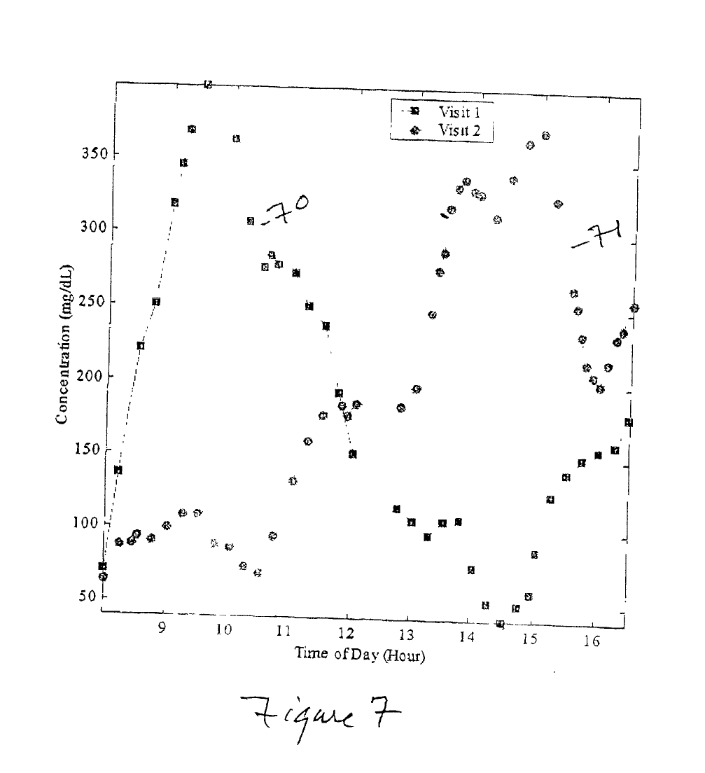

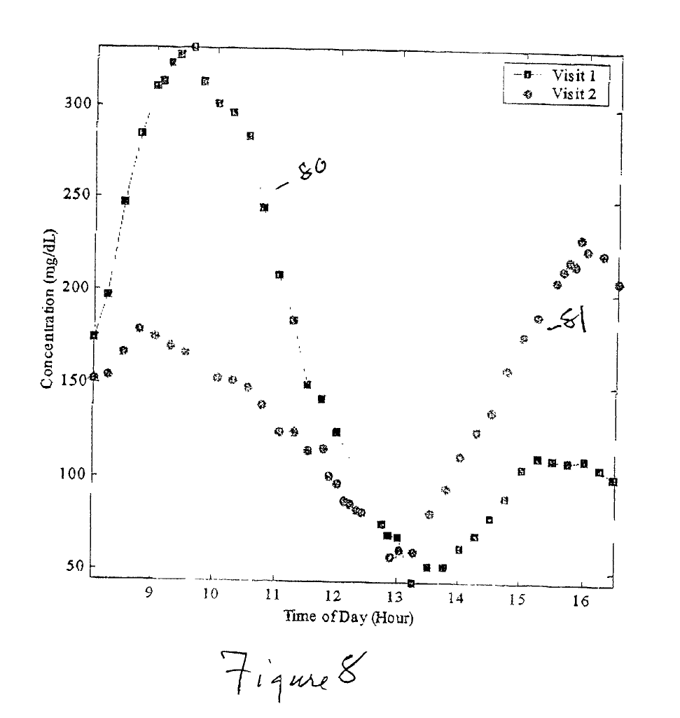

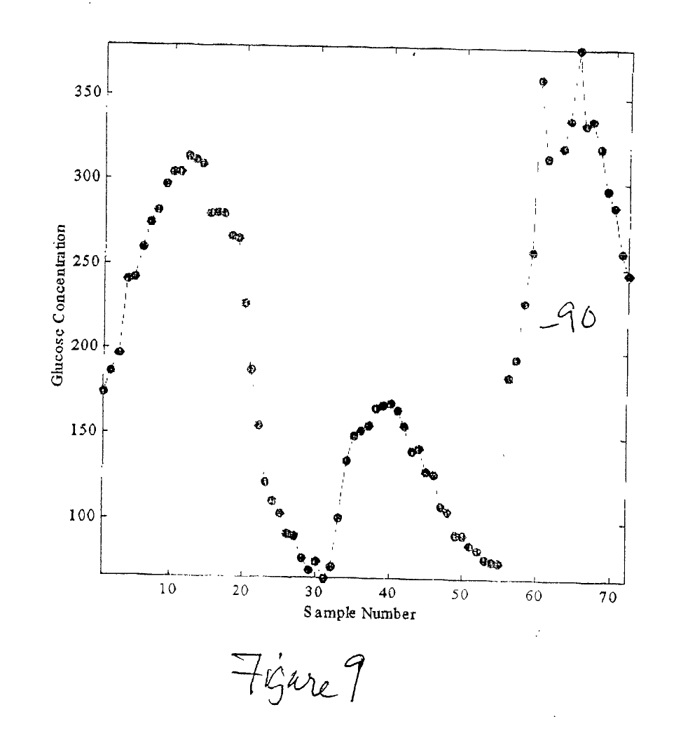

FIGS. 3 and 4 display the glucose profiles for each subject's calibration visit 1 and 2, respectively. The boldfaced curves represent the targeted glucose profiles 10, 11, for that visit. It is shown that the subjects' glucose levels were able to model the upward swing on both calibration visits. The increases were easily achieved with appropriate carbohydrate intake. The downward trends of the afternoons of calibration visit I and mornings of calibration 2 were achieved with less frequency than the upward trends. FIGS. 5 through 8 show the profiles of four single subjects. For each subject, the profiles for visit 1 50, 60, 70, 80 respectively and visit 2 51, 61, 71, 81 are imposed on each other.

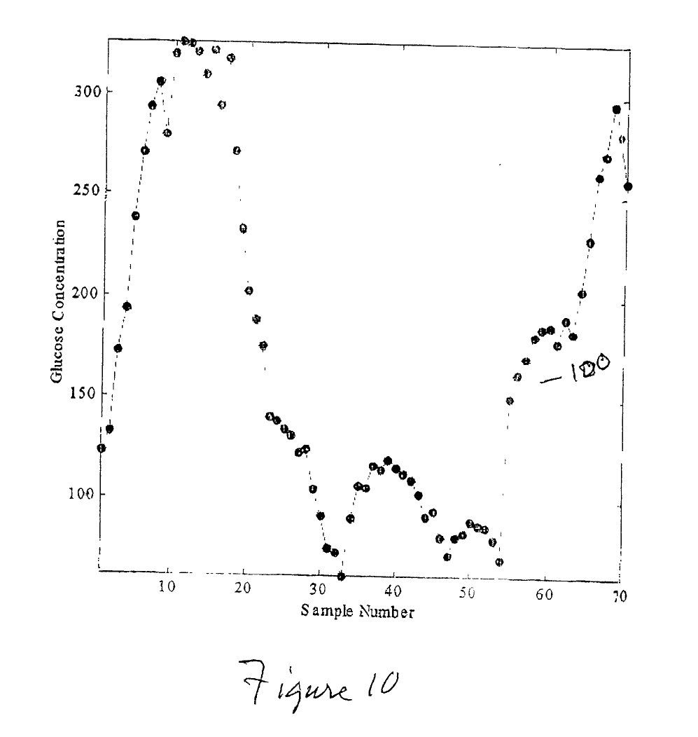

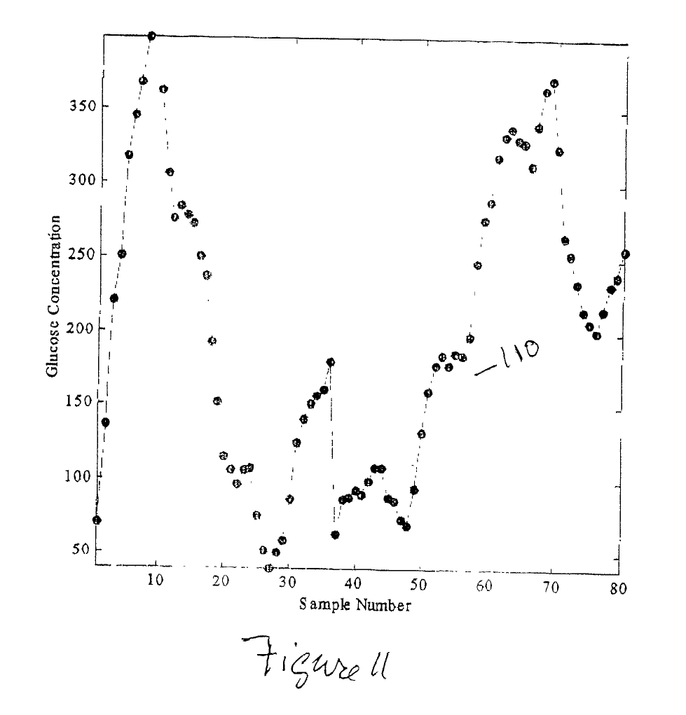

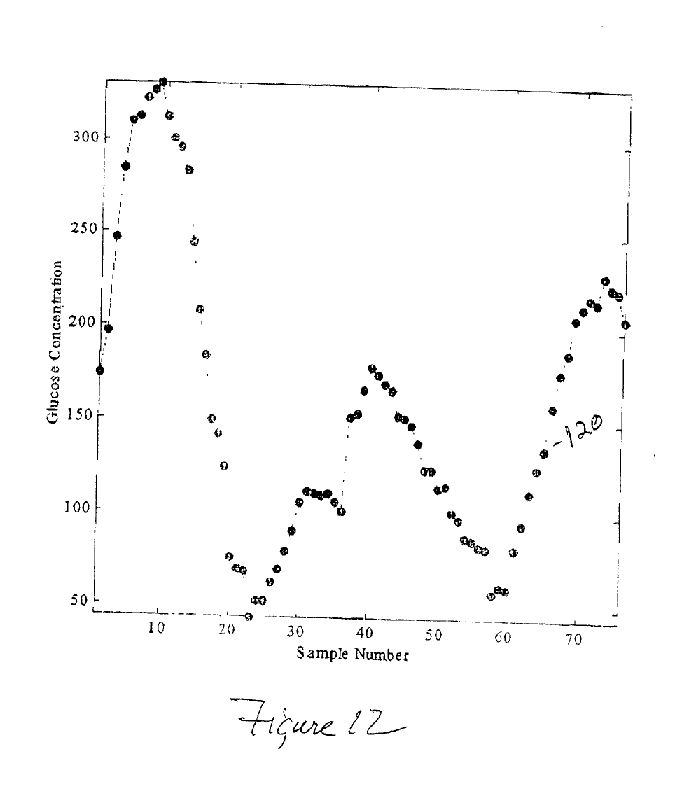

As previously indicated, the rates of change for the downward trend were often less than those for the upward trend toward the maximum, even with the administration of exogenous insulin. FIGS. 9-12 show visit 3 profiles 90, 100, 110, 120 for the same four subjects. For the visit 3 profiles, a more aggressive insulin-dosing regimen was employed to bring blood sugar levels down. It is apparent from the profiles that the more aggressive insulin-dosing regimen produces upward and downward rates of change that approximate each other more closely than those of visits 1 and 2.

The rate of change between the maximum glucose level and minimum glucose level was calculated for the first calibration visit (Table 2). This was calculated according to:

$$Rate of change = \frac{( max glucose ) - ( min glucose )}{( max time ) - ( min time )} . \qquad ( 2 )$$

The rate of change is expressed as milligrams per deciliter (mg/dl) over minutes. The rate of change is an indicator of a subject's capacity for the movement in blood glucose necessary to achieve the targeted glucose profile. The targeted glucose profile's rate of change is ±1.33 (mg/dl)/minute. For calibration visit one, the rate is a negative value, since it describes a downward trend. As Table 3, below, shows, three subjects (4, 5, and 6) had rates similar to that of the targeted profile.

Table 3 shows the percentage of the visit that it took to achieve a fluctuation from a maximum to a minimum in the case of visit 1, or a minimum to a maximum in the case of visit 2, calculated according to:

$$\% of visit = \frac{\begin{matrix}( time at max glucose value ) - \\ ( time at min glucose value )\end{matrix}}{ending time - initial time} * 100. \qquad ( 3 )$$

TABLE 3
Rate of change from maximum to minimum glucose
value and percent of visit spent fluctuating between maximum
and minimum glucose levels during calibration visits 1 and 2.

| | |
|---|---|
| Visit 1 | Visit 2 |

| | Rate of | | Rate of | |
|---|---|---|---|---|
| Subject | change | % of visit | change | % of visit |
| TARGET | −1.33 | 43.8 | 1.33 | 43.8 |
| 1 | −0.58 | 62.1 | 0.48 | 70.66 |
| 2 | −0.89 | 54.4 | 1.86 | 32.14 |
| 3 | −0.81 | 64.0 | 0.95 | 47.13 |
| 4 | −1.20 | 59.1 | 0.74 | 82.24 |
| 5 | −1.42 | 29.7 | 2.11 | 23.68 |
| 6 | −0.34 | 79.5 | 0.96 | 52.30 |
| 7 | −1.30 | 43.4 | 0.95 | 35.59 |
| 8 | −0.80 | 79.5 | 1.90 | 29.49 |
| 9 | −0.24 | 71.3 | 1.06 | 26.53 |
| 10 | −0.40 | 53.0 | 0.82 | 29.40 |

The visit percentage provides and indicator of the amount of time over the visit for the subject to fluctuate between the maximum and minimum of their glucose profile. According to the target, the subject should require only 43.8% of the visit to travel between a maximum and a minimum in order to achieve the desired glucose profile during the first calibration visit. All, except Client 5 and 7, took more time to move from the maximum to minimum glucose value, not allowing for enough time to start the upward trend at the end of the first calibration visit.

The results indicate that administering a calculated amount of carbohydrate can be used to achieve anti-correlated glucose patterns. Type 2 individuals are less sensitive to carbohydrate excursion and require two to three times the amount of carbohydrate of that of Type I individuals.

The invented formula, represented as Equation 1, also provides the clinician with a method of quantifying the amount of carbohydrate necessary to achieve a desired blood glucose excursion in a diabetic subject. The formula takes into account the required glucose level to be achieved, or the target, the current blood glucose level, or the starting value, and the sensitivity of the individual to carbohydrate.

‘X’ is a factor that serves as an index to carbohydrate sensitivity. The initial value is assigned by the clinician, according to type of diabetes and level of diabetes control, from a range of approximately 1 to 3, and is subsequently individualized to the subject. The amount of carbohydrate required to produce a target glucose excursion is calculated using a starting, generalized value of X, assigned by the clinician, as previously described. The diabetic subject then ingests the calculated amount of carbohydrate. Blood glucose values are measured at regular intervals until the subject's blood glucose values reach a maximum. The actual maximum and the target maximum are compared and an individualized value of X, X₁ is calculated according to:

$${X}_{i} = \frac{OBSERVED - STARTING}{CHO} , \qquad ( 4 )$$

where ‘OBSERVED’ represents the observed maximum, as contrasted with the target maximum. Thus, for an individual, assigned an initial X value of 2, who attained a maximum of 297 mg/dl following ingestion of an amount of carbohydrate calculated to produce a maximum of 350 mg/dl, the individualized value of X, X₁, would be calculated as 1.7. This calculated value can be used by the subjects to further enhance their diabetes management. It can be assessed that the Type I clients (4 and 8) had a much higher sensitivity to carbohydrates (2.10 and 3.09, respectively) than the other clients. Table 4 below provides the sensitivity factors and Carbohydrate quantities employed for visit one profiles.

TABLE 4
CHO intake and sensitivity factor utilized in visit one profiles

| | | CHO | Glucose |
|---|---|---|---|
| Subject | X | intake | excursion |
| 1 | 0.99 | 145 | 144 |
| 2 | 0.64 | 216 | 139 |
| 3 | 0.83 | 246 | 203 |
| 4 | 2.10 | 156 | 328 |
| 5 | 0.48 | 260 | 125 |
| 6 | 0.37 | 274 | 102 |
| 7 | 1.23 | 128 | 157 |
| 8 | 3.09 | 75 | 232 |
| 9 | 0.61 | 246 | 151 |
| 10 | 0.38 | 196 | 74 |

The calibration visits also provide an educational experience for the diabetic subjects. The test subjects indicate a greater awareness of the impact of carbohydrate foods on their blood glucose levels. Subjects who experience higher sensitivities in the morning may choose to move more of their carbohydrate food choices to the afternoon or evening, when their medication regimen may produce lower sensitivities. Furthermore, subjects report that their intake of carbohydrate is generally reduced, that they typically take smaller-sized portions of carbohydrate foods, and that nutritional information from food labels is more meaningful, all highly desirable outcomes in the management of diabetec conditions.

Furthermore, the invented formula and the individualized ‘X’ value may be used in the dietary management of any health condition where it is desirable to achieve and maintain an optimal glycemic profile. Those skilled in the art will appreciate other applications of the invented formula in general, along with applications of the general and individualized X values.

The absorption and, therefore, the activity of rapid-acting insulin are known to be highly individual. A further advantage of the invented methods is the capability of optimizing insulin injections relative to meal times. Review of blood test data generated during the calibration visits allows the individual's insulin response to be pinpointed easily. The time of injection is noted, and the point at which the glucose values begin to diminish is checked against the rate of change across intervals. When consistent patterns are observed, the onset of peak action can be verified.

Although the invention has been described herein with reference to certain preferred embodiments, one skilled in the art will readily appreciate that other applications may be substituted for those set forth herein without departing from the spirit and scope of the present invention. Accordingly, the invention should only be limited by the claims included below.

What is claimed is:

1. A method for shifting blood glucose level in an individual from a starting value to a target value, said method comprising the steps of:
calculating a required amount of carbohydrate to ingest to produce said shift according to a formula, said formula comprising:
$$CHO = \frac{TARGET - STARTING}{X} ,$$
where CHO represents said required amount of carbohydrate, and wherein X comprises an index representing said individual's sensitivity to carbohydrate;
ingesting said first required amount of carbohydrate by said individual; and
observing an actual shift in blood glucose value caused by ingesting said required amount of carbohydrate.

2. The method of claim 2, wherein said individual is a human subject.

3. The method of claim 2, wherein said carbohydrate to ingest is any of:
liquid food;
solid food; and
liquid and solid food.

4. The method of claim 2, wherein X comprises a generalized value.

5. The method of claim 4, wherein said generalized value is from the range of approximately 1 to 3.

6. The method of claim 4, wherein said blood glucose shift comprises a glucose excursion.

7. The method of claim 6, wherein said required amount of carbohydrate to produce a predetermined glucose excursion is calculated based on said exemplary value of X.

8. The method of claim 7, further comprising the step of individualizing said value of X to said individual based on an actual glucose excursion resulting when said individual ingests said required amount of carbohydrate according to:
$${X}_{i} = \frac{OBSERVED - STARTING}{CHO} ,$$
where ‘Observed’ represents an actual blood glucose value achieved following said ingestion of said first calculated amount of carbohydrate, and X₁ represents said individualized value of X.

9. The method of claim 8, further comprising the step of:
calculating a second required amount of carbohydrate using said individualized value of X wherein said second required amount comprises the amount of carbohydrate required to be ingested by said individual to effect said target glucose excursion.

10. The method of claim 9, further comprising the step of:
ingesting said second required amount of carbohydrate by said individual.

11. The method of claim 9, further comprising the step of:
achieving and maintaining an optimal glycemic profile based on said formula and said individualized value of X.

12. The method of claim 10, further comprising the step of:
generating an individualized calibration model for said individual for use in non-invasive methods of blood glucose determination employing spectroscopic instrumentation based on idealized anti-correlated glycemic profiles produced using said formula.

13. The method of claim 2, further comprising the step of:
using exogenous insulin to assist shifts between blood glucose levels.

14. A method of dietary management of an individual's glycemic profile, wherein an optimal glycemic profile is achieved and maintained, said method comprising the steps of:
calculating a required amount of carbohydrate to ingest to shift blood glucose level in said individual according to a formula, said formula comprising:
$$CHO = \frac{TARGET - STARTING}{X} ,$$
where ‘Target’ represents said target value, ‘Starting’ represents said first value and CHO represents said required amount of carbohydrate, and wherein X represents a generalized index value representing said individual's sensitivity to carbohydrate;
ingesting said required amount of carbohydrate by said individual;
observing the actual shift in blood glucose value caused by ingesting required amount of carbohydrate;
generating a value of X individualized to said individual; and
achieving and maintaining an optimal glycemic profile based on said formula and said individualized value of X.

15. The method of claim 14, wherein said individual is human.

16. The method of claim 14, wherein said carbohydrate to ingest is any of:
liquid food;
solid food; and
liquid and solid food combined.

17. The method of claim 14, wherein said generalized value of X is from a range of approximately 1 to 3.

18. The method of claim 17, wherein said blood glucose level shift comprises a glucose excursion.

19. The method of claim 18, wherein said required amount of carbohydrate to effect a predetermined glucose excursion is calculated based on said exemplary value of X.

20. The method of claim 19, wherein said generating step comprises:
individualizing X to said individual, based on an actual glucose excursion resulting when said individual ingests said first calculated amount of carbohydrate, according to:
$${X}_{i} = \frac{OBSERVED - STARTING}{CHO} ,$$
where ‘Observed’ represents an actual blood glucose value achieved following said ingestion of said first calculated amount of carbohydrate and X₁ represents said individualized value of X.

21. The method of claim 20, wherein said achieving and maintaining step comprises the steps of:
calculating a second required amount of carbohydrate to ingest to achieve and maintain said optimal glycemic profile based on said formula and said individualized value of X; and
ingesting said second required amount in divided portions over a predetermined time span.

22. The method of claim 14, further comprising the step of:
using exogenous insulin to assist in achieving and maintaining said optimal glycemic profile.

23. The method of claim 14, further comprising the step of:
generating an individualized calibration model for said individual for use in non-invasive methods of blood glucose determination employing spectroscopic instrumentation based on idealized anti-correlated glycemic profiles produced using said formula.

24. A method of predicting a required amount of carbohydrate to ingest to shift blood glucose level in an individual from a starting value to a target value, said method comprising the steps of:
providing said target value and said starting value;
calculating a difference between said values; and
calculating said required amount of carbohydrate by dividing said difference by a numerical index representative of said individual's sensitivity to carbohydrate according to:
$$CHO = \frac{TARGET - STARTING}{X} ,$$
wherein CHO represents said required amount and X represents said numerical index.

25. The method of claim 24, wherein said individual is a human subject.

26. The formula of claim 24, wherein said carbohydrate to ingest is any of:
liquid food;
solid food; and
liquid and solid food combined.

27. The method of claim 24, wherein X comprises a generalized value.

28. The method of claim 27, wherein said generalized value is from the range of:
approximately 1 to 3.

29. The method of claim 27, wherein said blood glucose level shift comprises a glucose excursion.

30. The method of claim 29, wherein said required amount of carbohydrate to produce a predicted glucose excursion is calculated based on said generalized value of X.

31. The method of claim 30, wherein said value of X is individualized based on an actual glucose excursion resulting when said individual ingests said first calculated amount of carbohydrate according to:
$${X}_{i} = \frac{OBSERVED - STARTING}{CHO} ,$$
where ‘Observed’ represents an actual blood glucose value achieved following said ingestion of said required amount of carbohydrate and X₁ represents said individualized value of X.

32. The method of claim 31, further comprising the step of achieving and maintaining an optimal glycemic profile based on said individualized value of X.

33. The method of claim 31, further comprising the step of producing idealized, anti-correlated glycemic profiles in an individual based on said formula and said individualized value of X, so that individualized calibration models may be generated for use in non-invasive methods of blood glucose determination employing spectroscopic instrumentation.

34. The method of claim 24, wherein shifts between glucose levels are assisted by the administration of exogenous insulin.
